(19) United States
(12) Patent Application Publication
(10) Pub. No.: US 2009/0299462 A1
(43) Pub. Date: Dec. 3, 2009

(54) ENDOVASCULAR GRAFT INCLUDING SUBSTRUCTURE FOR POSITIONING AND SEALING WITHIN VASCULATURE

(76) Inventors: Natalie V. Fawzi, Belmont, CA (US); Kimberly Barkman, San Mateo, CA (US); Robin W. Eckert, San Jose, CA (US); Arnold M. Escano, Santa Clara, CA (US); Rodney H. Reinhardt, Newark, CA (US); Robert A. Vincent, Union City, CA (US)

Correspondence Address: BROOKS, CAMERON & HUEBSCH, PLLC, 1221 NICOLLET AVENUE, SUITE 500, MINNEAPOLIS, MN, 55403, US

(21) Appl. No.: 12/470,632

(22) Filed: May 22, 2009

Related U.S. Application Data
(62) Division of application No. 10/445,721, filed on May 27, 2003, now abandoned.

(51) Int. Cl. A61F 2/06

(52) U.S. Cl. 623/1.13; 623/1.23; 623/1.35

(57) ABSTRACT

An endovascular graft having an improved positioning mechanism capable of positioning and securing a bifurcated graft into a bifurcated vessel described. The graft can include a sleeve affixed to graft that is used in combination with a contralateral wire loop for placement of the graft within vasculature. The graft may include a structure for post deployment positioning into the bifurcated aneurysm area. Furthermore, the endovascular graft may be configured to form a sealing pocket that expands with induced fluid pressure and prevents fluid leaks at an attachment site.

### BACKGROUND OF THE INVENTION

The present invention relates generally to vasculature repair and more particularly to devices for accomplishing positioning and securement of a repair device at an interventional site.

It is well established that various fluid conducting body or corporeal lumens, such as veins and arteries, may deteriorate or suffer trauma so that repair is necessary. For example, various types of aneurysms or other deteriorative diseases may affect the ability of the lumen to conduct fluids and, in turn, may be life threatening. In some cases, the damage to the lumen is repairable only with the use of prosthesis such as an artificial vessel or graft.

For repair of vital lumens such as the aorta, surgical repair is significantly life threatening or subject to significant morbidity. Surgical techniques known in the art involve major surgery in which a graft resembling the natural vessel is spliced into the diseased or obstructed section of the natural vessel. Known procedures include surgically removing the damaged or diseased portion of the vessel and inserting an artificial or donor graft portion inserted and stitched to the ends of the vessel which were created by the removal of the diseased portion. More recently, devices have been developed for treating diseased vasculature through intraluminal repair. Rather than removing the diseased portion of the vasculature, the art has taught bypassing the diseased portion with a prosthesis and implanting the prosthesis within the vasculature. An intra arterial prosthesis of this type has two components: a flexible conduit, the graft, and the expandable framework, the stent (or stents). Such a prosthesis is called an endovascular graft.

It has been found that many abdominal aortic aneurysms extend to the aortic bifurcation. Accordingly, a majority of cases of endovascular aneurysm repair employ a graft having a bifurcated shape with a trunk portion and two limbs, each limb extending into separate branches of vasculature. Currently available bifurcated endovascular grafts fall into two categories. One category of grafts are those in which a preformed graft is inserted whole into the arterial system and manipulated into position about the area to be treated. This is a unibody graft. The other category of endovascular grafts are those in which a graft is assembled in-situ from two or more endovascular graft components. This latter endovascular graft is referred to as a modular endovascular graft. Because a modular endovascular graft facilitates greater versatility of matching the individual components to the dimensions of the patient's anatomy, the art has taught the use of modular endovascular grafts in order to minimize difficulties encountered with insertion of the devices into vasculature and sizing to the patient's vasculature.

Although the use of modular endovascular grafts minimize some of the difficulties, there are still drawbacks associated with the current methods. Where it is desirable to repair vasculature with a device that is assembled in situ, it can be difficult to accomplish positioning various components of the repair device within the diseased vessel. Moreover, attachment systems typically used for anchoring modular grafts and unibody grafts to a vessel wall can form improper seals and result in fluid leaks. A reoccurring difficulty relates to exposing certain of the modular junction attachment sites to continuous blood flow.

Other drawbacks associated with endovascular grafts involve providing components having a secure attachment to the main graft. The stitching pattern sewing a component to the graft material should be safe, such that if one suture connection is severed the repair device will remain secured.

To provide consistency with the common usage of terms used in the medical surgical arts in the United States, the terms “proximal, distal, inferior and superior” are used with a certain regularity within the present specification. “Proximal” refers to parts of the system, such as catheters, capsules and wires, which are closest to the user and closest to that portion of the system lying outside or exterior of the patient. “Distal” refers to the point farthest from the user and typically most interior of the corporeal lumen. The term “superior” refers to a location situated upstream of the flow of blood and is used herein in description of the graft and attachment system. “Inferior” refers to the point situated downstream of the flow of blood and again is used herein with reference to the graft and attachment system.

A typical procedure used with the described invention uses a “femoral approach.” This term describes an application which begins with an incision in the femoral artery. Similarly, the described invention may be used in an “iliac approach” which begins with an incision in the iliac artery. Using the terminology defined in the previous paragraph, the distal tip of the system may be inserted into the femoral artery and advanced upstream into the iliac artery and the abdominal aorta. Thus, the more distal portions of the system reside upstream of those portions described as more proximal. Furthermore, in the described procedure, the superior portions of the graft will permanently reside in the abdominal aorta, while the inferior portions will reside in the iliac arteries.

The femoral delivery approach for bifurcated grafts has its limitations. If the bifurcated graft is deployed close to the natural bifurcation of the aneurysm, there is potential that the inferior members will need to take a sharp bend in order to conform to the aortic anatomy. Positioning the bifurcated graft, using this approach, has resulted in kinking and twisting of the inferior graft members. These limitations may result in patency problems, and added stress to the sutures holding the implant components together.

The terms “ipsilateral” and “contralateral” typically refer to opposing portions of a corporeal lumen having symmetric right and left sides. “Ipsilateral” refers to those portions residing on the same side through which the grafting system enters the corporeal lumen, while “contralateral” refers to the opposite portions. Therefore, this distinction is dependent on whichever side (right or left) the physician decides to insert the grafting system. The portions of the grafting system which reside or operate within the symmetric vessels of the corporeal lumen use the same terminology. For example, the physician may insert the grafting device into the ipsilateral femoral artery, advance the device through the ipsilateral iliac artery and into the abdominal aorta. Then the device can be manipulated downstream into the contralateral iliac artery.

Accordingly, there exists a need for methods or devices which overcome or tend to minimize the challenges associated with positioning repair devices within bifurcated vasculature. The present invention addresses these and other needs.

### SUMMARY OF THE INVENTION

Briefly and in general terms, the present invention is directed towards repairing vasculature. More particularly, the present invention includes a system that is configured to accomplish intraluminal repair of defects such as aneurysms found in blood vessels. In one or more aspects, the present invention is directed at positioning a modular bifurcated graft within vasculature. In other aspects, the present invention is concerned with providing a sealing member at the attachment sites of a graft or repair device.

In one embodiment of the present invention, a sleeve is affixed to the inside of the graft bifurcation or crotch of a bifurcated graft, and assists in positioning the graft and its components within vasculature. An associated grafting system further includes a contralateral guide wire having a hook or bulbous portion on a terminal end of the guide wire. The hook or bulbous portion facilitates the snaring of the contralateral guide wire with a snare loop. The graft sleeve provides a pathway for the contralateral guide wire through the graft such that a physician may manipulate the contralateral guide wire to position the bifurcated graft at a repair site. Once the modular graft is positioned at the repair site, leg extensions may be assembled to the graft ipsilateral and contralateral leg stumps.

In another embodiment, an endovascular graft includes a graft pocket that radially expands in response to fluid pressure. The expanded graft pocket forms a seal at an attachment site or at non-uniform connection areas and redirects blood flow through the graft.

In a further embodiment of the present invention, an improved stitching pattern for attaching graft components is provided. The improved stitching pattern involves at least two double loop knots and at least two suture loops around structure to be attached to a graft, the structure being anchored with a running stitch having threaded loops and double loop knots. The stitching pattern provides a secure connection if one portion of the suture is severed or damaged.

In yet another embodiment of the invention, a sealing member is configured to radially surround the graft member attachment sites, wherein the sealing member is a tuft configured to assist blood clotting and induce endovascular tissue growth. One aspect of the sealing member is embodied in a tufted material formed of a polyethyleneterephthalate (PET) suture that is stitched circumferentially in an in-and-out pattern forming suture loops around the graft member attachment site, wherein the suture loops provide a surface for blood clotting and promotes tissue growth.

A second aspect of the sealing member is embodied in a tufted PET fabric formed from a non-woven web of loose fibers attached to the graft member walls by a suture thread, wherein the non-woven web has an in-air thickness of approximately 0.01 in. and a compressed thickness in the range of 0.007 in. to 0.008 in., and a width of approximately 5 cm. The non-woven tufted web provides a continued circumferential surface around the attachment member to assist in blood clotting of leaks and promoting tissue growth.

In still another embodiment of the present invention, the graft system includes a mating structure that releasably attaches the ipsilateral member and the contralateral members of a bifurcated graft, wherein the members are attached during deployment, and separated after deployment, thus allowing post-insertion positioning. The inferior members or limbs of a graft are connected together to improve control, stability, and column stiffness of the graft when accessing the contralateral artery.

In one aspect, the mating structure includes a release wire that is releasably threaded through a plurality of suture loops affixed to the ipsilateral member and contralateral member and secures the members together, wherein the removal of a release wire separates the graft members allowing the bifurcated graft to conform to a vessel bifurcation.

In a second aspect, the mating structure includes a suture material releasably configured to form a running stitch pattern that attaches the ipsilateral member and contralateral member. The suture begins at the graft bifurcation and is stitched in-and out through the ipsilateral member and contralateral member, a release wire being configured to disengage the members, thereby allowing positioning of the graft members at a vessel bifurcation.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 is a partial cross-sectional view, depicting a bifurcated graft with a sleeve positioning mechanism disposed about a contralateral guidewire facilitating the snaring of the contralateral guidewire by a snare device;

FIG. 2 is a partial cross-sectional view, depicting a modular bifurcated graft placed at a bifurcation with a sleeve positioning mechanism disposed about a contralateral guide wire and the deployment of the contralateral leg extension;

FIG. 3 is the partial cross-sectional view of FIG. 2, further depicting the sleeve facilitating the assembled leg extension;

FIG. 4 is a partial cross-sectional view, depicting a bifurcated graft implanted at a bifurcation with an attachment system attached to a main tubular member via a double loop knot stitching pattern and incorporating a graft pocket;

FIG. 5 is an elevational view of a portion of an endovascular graft incorporating a graft pocket;

FIG. 6A is an enlarged plan view of the stitching pattern shown in FIG. 4;

FIG. 6B is an enlarged plan view of eyelets attached to the inside of a graft wall;

FIG. 6C is an enlarged plan view of eyelets stitching pattern near the edge of a graft;

FIG. 7 is a side elevational view of a graft device, wherein a sealing member tuft loop is depicted;

FIG. 8 is a side elevational view of a graft device, wherein a sealing member tuft web is depicted;

FIG. 9 is a perspective view, depicting a modular bifurcated graft with ipsilateral and contralateral members mating structure having a plurality of loops and a release wire;

FIG. 10 is a partial cross-sectional view, depicting the modular bifurcated graft of FIG. 9 having separated ipsilateral members and being deployed within vasculature;

FIG. 11 is a perspective view, depicting a modular bifurcated graft with the ipsilateral and contralateral members mating structure having a suture running stitch securing the members together;

FIG. 12 is a partial cross-sectional view, depicting the modular bifurcated graft of FIG. 11 having separated ipsilateral members and being deployed within vasculature;

FIG. 13 is a partial cross-sectional view of FIG. 10, depicting a contralateral leg extension; and

FIG. 14 is a partial cross-sectional view of FIG. 12, depicting a contralateral leg extension.

### DETAILED DESCRIPTION OF THE PREFERRED EMBODIMENTS

As shown in the drawings and for the purpose of illustration the invention is embodied in an endovascular graft for repairing vasculature. A positioning mechanism is provided for facilitating the positioning of a graft within vasculature. The graft may include a sealing mechanism and attachment mechanisms to secure the graft within the vasculature. One of the disclosed features involves the use of a sleeve incorporated into the graft which is used in combination with a wire for placement of the graft across a vascular bifurcation such as the aortic bifurcation. Additionally, the graft includes a self-sealing means that compensates for oversizing of a vessel wall. The superior and inferior graft portions may be provided with improved leak tight sealing tufts. Furthermore, the graft may include a pattern for stitching a stent or other structure to members of a graft for securing the members together.

Those skilled in the art will recognize many of the disclosed components can be described by various terms. For example, the parts of the bifurcated graft may be referred to as superior and inferior members as well as upstream and downstream ducts or as distal and proximal extremities. The attachment systems are also referred to as expandable anchors which is descriptive of how the systems operate. The delivery components include tubular devices known as catheters in many different configurations. There exists a main delivery catheter for delivery of the entire system as well as secondary catheters which are used within the ipsilateral and contralateral blood vessels. The use of particular terminology herein is not intended as a limitation, rather terminology is intended to encompass the varied references known to those of skill in the art.

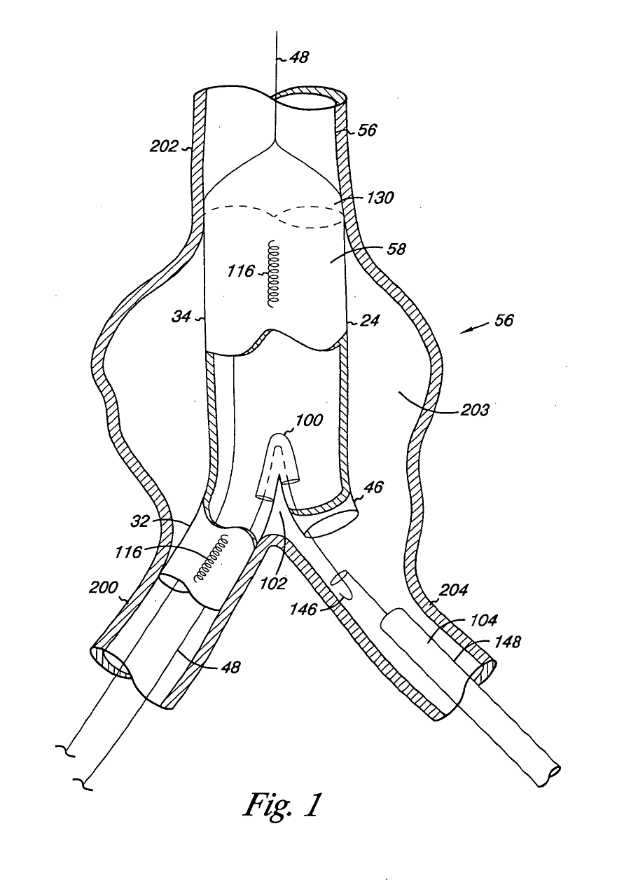

With reference to FIGS. 1-3, in one aspect, a modular graft 24 is shown embodied in a bifurcated tubular prosthesis having superior and inferior extremities. However, it is to be recognized that the various inventive aspects described herein can be applied to any tubular graft or medical device where positioning and secure placement is a concern. The superior member 34 of the graft 24 includes a main tubular member which bifurcates into an ipsilateral tubular leg and a contralateral leg stump which define the inferior extremities of the graft. It is to be recognized, however, that both the ipsilateral and contralateral legs can be defined by stumps. For clarity, the two tubular legs are referred to as the ipsilateral inferior member 32 and the contralateral inferior member 46.

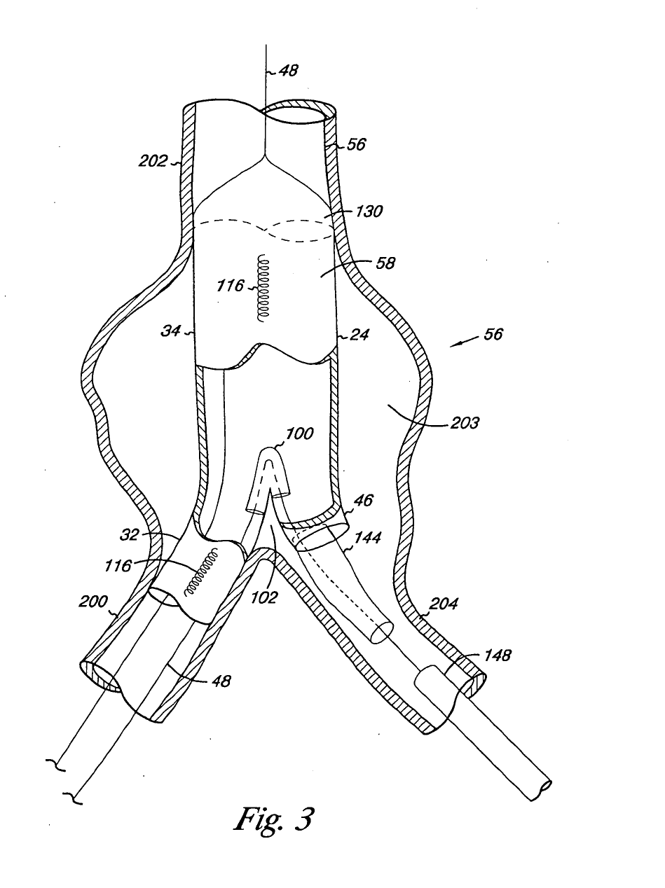

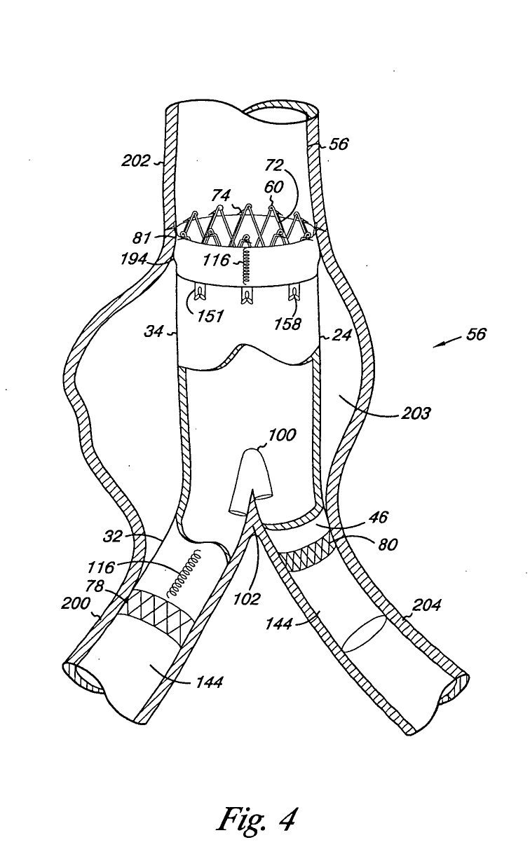

The modular graft 24, as shown in FIG. 1, is an expandable, collapsible and flexible intraluminal vascular bifurcated structure for implanting in a body vessel or corporeal lumen 56. The graft includes a deformable main tubular member 34 which bifurcates into an ipsilateral tubular member 32 and a contralateral tubular member 46. The main tubular member 34 and inferior tubular members 32, 46 each are formed of a graft wall 58 allowing fluid communication between the superior and inferior ends 32, 46 of the bifurcated graft 24. As depicted in FIG. 3, a graft leg extension 144 may be attached to the contralateral tubular member 46, likewise, a leg extension may be attached to the ipsilateral tubular member, see FIG. 4.

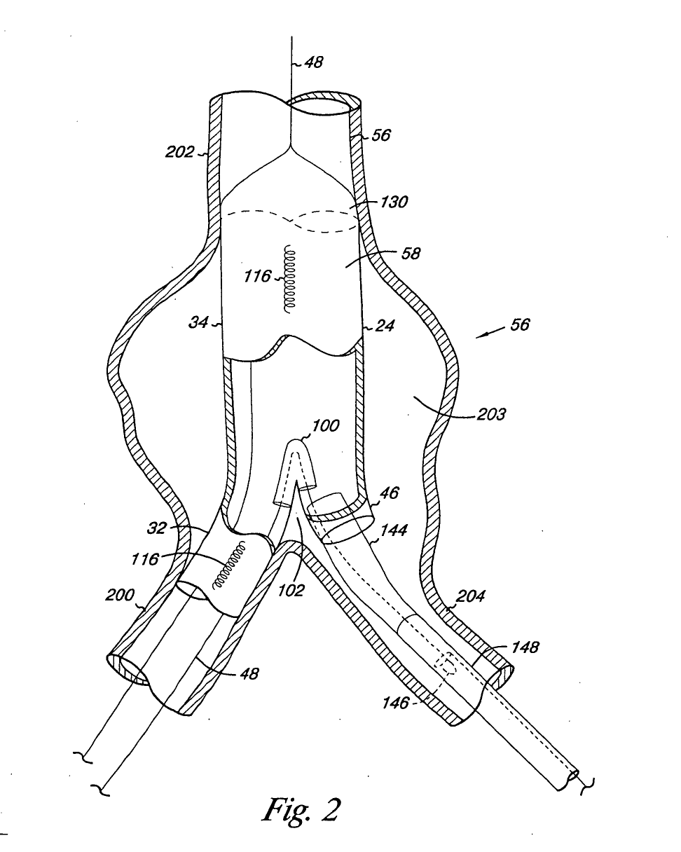

In one preferred embodiment of the present invention, as shown in FIGS. 1 and 2, the substructures employed to facilitate positioning the contralateral inferior member 46 within a contralateral iliac artery includes a sleeve 100 affixed to the graft bifurcation 102, an elongate positioning mechanism or contralateral guide wire 48, a contralateral catheter 148 and a contralateral snare loop device 104. The bifurcated graft sleeve 100 is affixed inside the graft bifurcation or crotch 102 though the sleeve 100 can be placed anywhere on a graft or other medical device. Preferably, the graft sleeve 100 is sized to slidably receive the guide wire 48 such that a physician may manipulate the guide wire 48 to place the bifurcated graft into position, for example to treat an AAA. It is contemplated that the guide wire 48 is slid inside the sleeve prior to deployment of the bifurcated graft within the corporeal lumen, however, the sleeve 100 can also be accessed in vivo.

The sleeve 100 is affixed in the crotch 102 of the graft 24 starting at the ipsilateral member 32 and extending across the crotch to the contralateral member leg 46. The sleeve 100 may be formed as an integral part of the graft 24 or can be affixed to the crotch bifurcation 102 of the graft wall 58 of the bifurcated graft 24 by any suitable means such as a polyester suture material or woven as an integral part of the graft material. The sleeve may be affixed with one or more sutures. The sleeve is configured of a flexible material, that may be the same material as the bifurcated graft or can embody any biocompatible material. In particular, the sleeve may be a fluid tight, material manufactured from a polytetra-fluoroethylene or a polyester fiber made from polyethylene terephthalate (PET). The sleeve can be any length and can extend the length or beyond the contralateral and ipsilateral limbs.

The sleeve 100 may further include a pressure sensing means (not shown) configured to measure the pressure induced by the graft on the aortic bifurcation of the aneurysm. Other sensors can be placed at or near the sleeve 100 to monitor other conditions such as flow.

The elongate positioning mechanism 48 can be formed by a conventional guide wire or other member embodying structure well suited for advancement within vasculature and can include a hook 146 (FIG. 1) formed on a terminal end thereof. The hook 146 can be replaced by a bulbous or enlarged portion for particular applications. This hook or bulbous portion facilitates the snaring of the positioning mechanism or guide wire 48 by an appropriate device inserted from the contralateral iliac artery. This device may then be used to position the contralateral member 46 of the graft into the contralateral iliac artery and withdraw the proximal end of the guide wire 48 through the contralateral femoral artery. This allows for the manipulation and positioning of the graft 24 through use of both the guide wire 48 and the snare device 104. This arrangement can also provide a platform for delivering other components to an interventional site such as graft extensions or other medical devices.

An attachment system is secured to the superior end of the main tubular member 34 as well as to the inferior ends of each of the tubular legs 32, 46. The superior attachment system 60 (See FIG. 4) secured to the superior member may be provided with wall-engaging members 74 which are retracted or covered during delivery. The attachment system 78 may be attached to the ipsilateral leg 32 to secure the graft while inserting additional support structures in the form of expandable stents to extend the length of the contralateral leg either along an interior or exterior of the graft 24. A balloon catheter assembly 130 (FIG. 3) may be included for expansion of the attachment systems or to aid in implantation. The attachment systems may be balloon expanded or self-expanding and can be attached to the exterior or interior of the graft 24. Release wires or capsules (not shown) can be employed to keep the attachment systems in a compressed condition until the bifurcated graft 24 is appropriately positioned.

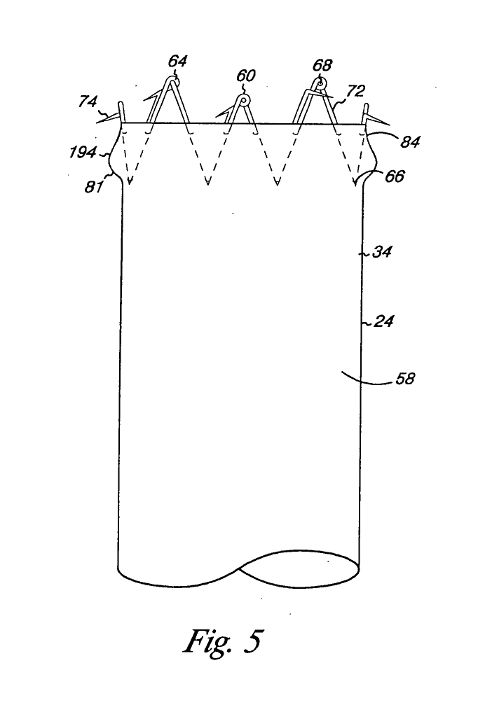

The superior attachment system 60 (See FIG. 4), can be expanded via a balloon member 130 or allowed to self-expand. The balloon member 130 can additionally be used to force the attachment system and a plurality of outwardly disposed wall-engaging members 74, if present, into the wall of the vasculature 202. As shown in FIGS. 4-5, wall-engaging members 74 are preferably secured to the legs 72 of the superior attachment system 60 in the vicinity of the outer apices 64 by suitable means such as a weld. Alternative configurations for the attachment system as well as the wall-engaging members may be used. In the embodiment shown, the wall-engaging members 74 are bent as hooks and are preferably sharpened to provide conical tips. The wall engaging members should have a length which is sufficient for the tip to penetrate into and perhaps through the corporeal lumen wall. The superior attachment system 60 and wall-engaging members 74 may be formed from any suitable, corrosion resistant wire material. One such material is ELGILOY™ which is a cobalt-chromium-nickel alloy manufactured and sold by Elgiloy of Elgin, Ill.

Referring to FIGS. 4-5, the superior attachment system 60 is secured adjacent a superior end 81 of the main tubular member 34. The superior attachment system may be formed of a plurality of apices with the outer apices 64 and inner apices 66 of the superior attachment system 60 possibly being formed with helical torsion springs 68 and securely attached within the main tubular member 34. The expanded attachment system is configured to facilitate in providing a self sealing graft pocket 194 that excludes blood flow from the repaired vasculature.

In one embodiment, the graft 24 includes a graft pocket 194 that is radially expanded when blood flows into the graft, thereby forcing the graft pocket 194 to create a leak tight seal against the vasculature wall below the wall-engagement members 74 of the prosthesis (See FIGS. 4 and 5). The graft pocket 194 can be formed by weaving the graft 24 to include an annular portion having an increased diameter. The graft pocket 194 can extend completely around a circumference of the device or can define discrete pockets thereabout. Moreover, the graft pocket 194 can be formed of the same or different material of the graft. As such, it is contemplated that the graft pocket 194 can be defined by expandable or self-expanding structure. It is also to be recognized that the graft pocket 194 is configured to occupy spaces between the graft and a lumen into which the graft is implanted and thus can form any portion of the graft or for that matter any medical device. Accordingly, although the description describes configuring the superior end of a graft with a graft pocket 194, such structure may be applied to the inferior members or other portions of the graft as well.

When placed within a blood vessel, the portion of the graft 24 that is directly pressed against the vessel wall 202 by a wire frame or attachment system forms a seal that assists in the prevention of fluid leaking around the end of the graft 24. Since the wire frame is continuous, the portion of the graft that is pressed directly against the vessel wall should in most cases be continuous. It is therefore the relieved portions of the graft, not pressed against the vessel, which are most vulnerable to leaks. Leaking is more likely to occur if the vessel at an interventional site is deformed or irregular in shape. For example, the graft 24 may have a slightly larger diameter than the inner dimension of the vessel 202 or the vessel wall may not be smooth. In such circumstances, pleats in the graft 24 are sometimes formed between the struts 72. Another factor that increases the likelihood of pleating is the pulsing of the blood vessel during the cardiac cycle. When the blood vessel is contracted, pleating may be mildly accentuated.

In the disclosed embodiment, the diameter of the circumferential graft pocket 194 may be one to six millimeters larger than the diameter of the main tubular member 34. It should be noted that the expandable attachment system frame need not be attached to the pocket section of the prosthesis, thereby allowing the graft pocket to move freely.

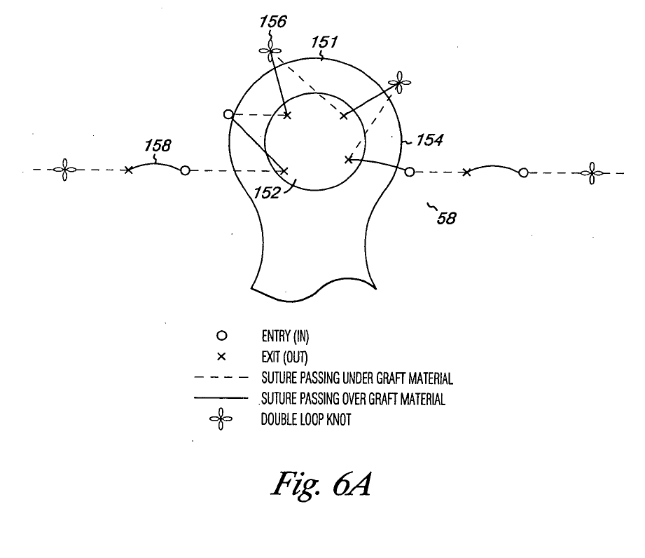

In the embodiment wherein the attachment system 60 forms structure separate from the graft 24, connection to the graft 24 can be accomplished by sewing suture material 158 into and out of the graft wall 58 and by forming at least two knots and two loops around a portion of the attachment system 60 such as an eyelet 151 of the attachment system 60 and then securing each side of the eyelet 151 with one threaded loop and an anchoring double loop knot 156. This pattern for stitching an eyelet to the graft material, as shown in FIGS. 6A-6C, provides security in case a single suture is severed or damaged. The security is based on the location of knots and the number of loops in the stitching pattern.

The attachment prosthesis may include a plurality of eyelets 151 affixing the prosthesis and the graft 24, as shown in FIG. 4. The stitching pattern at each eyelet 151 involves forming a double loop knot 156 in the graft material to anchor a first side of the eyelet 151, threading the suture thread 158 into and out of and into the graft wall again, and passing the suture thread under the eyelet 151 wherein the suture exits the graft material on the eyelet inner side 152. Next, the suture is threaded over the eyelet outer surface 154 into the graft material forming one complete loop around the first side of the eyelet 151, a second loop is formed by threading the suture under the eyelet from the outside into the eyelet inner side, and the suture is again passed over the eyelet surface, thereby completing a second loop and thereby anchoring the eyelet by forming a double loop knot 156 at the eyelet outer side. Further, the suture is threaded from the knot into the graft material, passing from the eyelet outer side into the graft material at the eyelet inner side, passing over the eyelet surface completing a third loop around the eyelet wherein a second anchor is formed with another double loop knot 156. From the knot the suture is threaded into the graft wall, passing under the eyelet 151 exiting the graft wall at the graft inner side, passing over the eyelet surface entering the graft material at the eyelet outer side, therein completing the fourth loop around the eyelet. From the eyelet second side the suture is threaded from the outer side into and out of the graft material twice, forming one and one-half loops which are anchored by a double loop knot 156. The pattern can be adjusted for stents attached to the inside or outside of a graft (See FIG. 6B), and for eyelets attached near the edge of a graft (See FIG. 6C) or in the body of the graft.

Those skilled in the art will appreciate that the improved stitching pattern described above may be used to affix the attachment system 60 to graft material 58 via eyelets formed at the proximal apices of the attachment system, as well as other prosthesis attachment devices not mentioned herein. Thus, it is contemplated that the ipsilateral and contralateral attachment system 78, 80 or other components can be similarly affixed to the graft 24.

Preferably the ipsilateral attachment system 78 and the contralateral attachment system 80 are disposed within the ipsilateral inferior member 32 and the contralateral inferior member 46, respectively. However, these attachment systems as well as the superior attachment system can be affixed to an exterior of the graft 24. The attachment systems should be arranged such that upon implantation, a superior end of the ipsilateral attachment system 78 and the superior end of the contralateral attachment system 80 are located proximal to the crotch 102 of the bifurcated graft 24, as shown in FIG. 4. Although shown as braided structures, the ipsilateral and contralateral attachment system can assume any configuration. As a braided type of endoprosthesis often decreases in length while expanding in diameter, the preferred arrangement upon implantation is positioned appropriately before full deployment. A simple calculation of the amount of shortening due to the desired expansion will allow the endoprostheses 78, 80 to be appropriately placed during manufacture to allow for the proper positioning upon expansion. One preferred embodiment is to use an endoprosthesis which has a maximum diameter larger than the maximum diameter of the tubular member, such as using a 14 mm diameter (relaxed state) endoprosthesis with a 13 mm diameter maximum tubular member.

The sizing of the bifurcated graft 24 may be performed on a patient by patient basis, or a series of sizes may be manufactured to adapt to most patient's needs. For the repair of an aortic aneurysm, the length of the bifurcated graft 24 is selected so as to span at least one centimeter superior and one centimeter inferior of the repair site, whereby the attachment systems and graft can contact healthy tissue of the vessel on both sides thereof. Thus, the bifurcated graft 24, not including the attachment systems, should be at least two centimeters longer than the site being repaired. During the pre-implant fluoroscopy procedure conducted in connection with AAA repair, a conventional pig tail angiography catheter is used to determine the locations of the renal arteries to ensure the renal arteries will not be covered by the implanted graft. Likewise, determining the location of the internal iliac arteries ensures that they will not be covered by the solid portion of the implanted graft 24. Also, the diameter of the main tubular member 34 is selected by measuring the corporeal lumen which will receive the graft by conventional radiographic techniques and then selecting a graft with a main tubular member having a diameter the same as measured and preferably at least one millimeter larger than that measured.

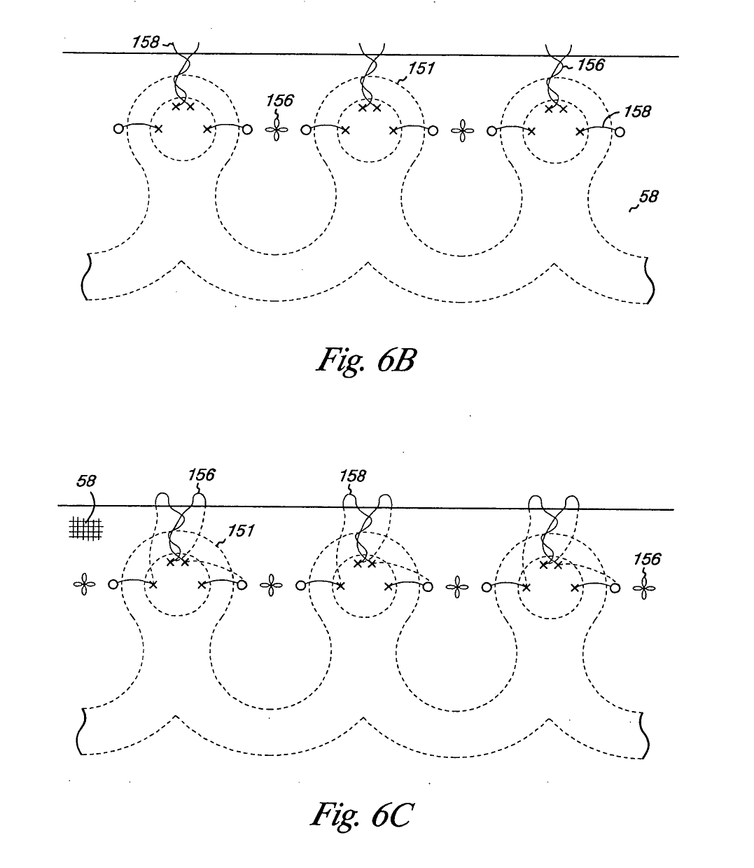

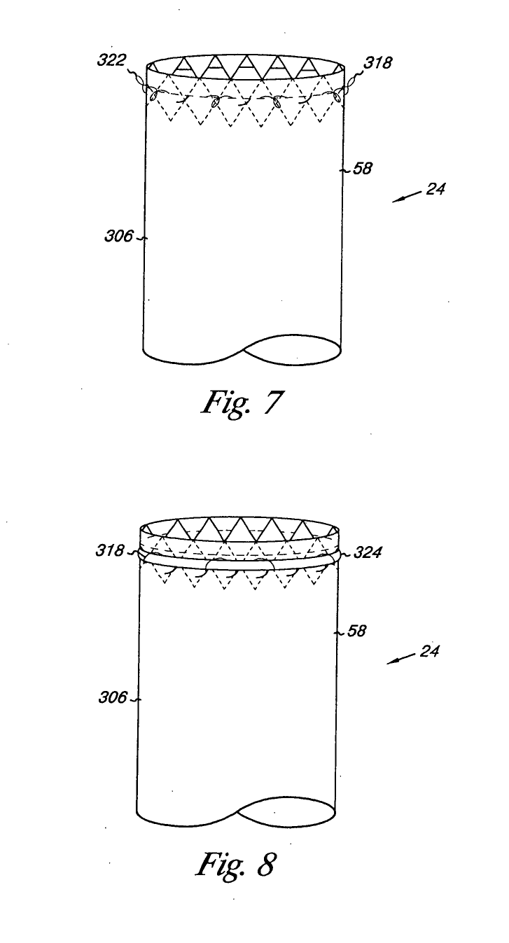

The further prevention of leaks can be accomplished by texturing the outside of the graft 24 with a plurality of filaments or fibers that are spun, woven, knotted, pressed or otherwise loosely associated to form a puffed textured filler that can be sewn to or affixed to the outside of the graft proximal to the end of the graft. The filler of the embodiments illustrated in FIGS. 7 and 8 includes stitches of a biocompatible synthetic material called tufts 318.

As shown in FIGS. 7 and 8, a graft 24 may include sealing members that are formed from tufted material 318, which may induce tissue growth, and which is affixed to the outer walls 306 of the graft 24. When the graft is deployed in a diseased vessel, the tufted material 318 operates to fill spaces between the vascular wall and the tubular member, thereby substantially forming a seal. Where there is a continuous blood flow or leak over a tuft near the attachment site of two joining implants sections, increased tissue growth and/or blood clotting will aid in the sealing of the union. In addition, the clotting and/or tissue growth may decrease the potential for an endoleak. In one form of the improved graft 24 having a tufted sealing member 318, the tufted sealing member 318. is located on the outer surface 306 of the graft 24 between members defining the attachment system (See FIG. 7).

In a preferred embodiment, the tuft is formed of continuous polyethylene terephthalate (PET) suture stitched circumferentially about a graft 24. As shown in FIG. 7, the suture stitching pattern would alternate in-and-out of the attachment system forming a small 2-2.5 mm loop 322 staggered evenly around the attachment site. The PET loops 322 of the tuft provide a surface to which blood may clot to fill the space and prevent further leaks.

In a another preferred embodiment, a tufted layer of PET fabric made from a non-woven web of loose fibers is simply attached to the outer wall 58 of the graft 24 by stitching the fiber on to the wall of the tubular member (See FIG. 8). Under magnification the non-woven PET fabric reveals loose openings between fibers, similar to a velour graft, but porous enough to allow blood flow through and around the layered material. The non-woven PET web 324 has an in air thickness of approximately 0.01 in., the compressed thickness may be approximately 0.007-0.008 in., and the width of the fabric is approximately 5 cm wide.

The non-woven tufted web 324 provides a continued circumferential sealing surface around the graft 24 to assist in blood clotting of leaks. A second benefit of both the tufted web and the tuft loop embodiments becomes apparent once the graft 24 has been in place for a considerable period of time and tissue begins to build up along the wall of the blood vessel. The tissue growth that builds up to the side of the graft from the blood vessel wall further anchors ends of the graft 24 to vasculature. For certain applications, the tufted material may be impregnated with a thrombogenic substance to induce coagulation and tissue growth.

Those skilled in the art will appreciate that the tufted systems described above may be formed of other suitable materials. The tuft sealing member may be affixed to non-bifurcated grafts or other medical devices as well. Another way to attach the circumferential tufts or tufted fabric layers is through ultrasonic welding using specific spot welds less than 0.01 in. at precise locations between the tufts and graft.

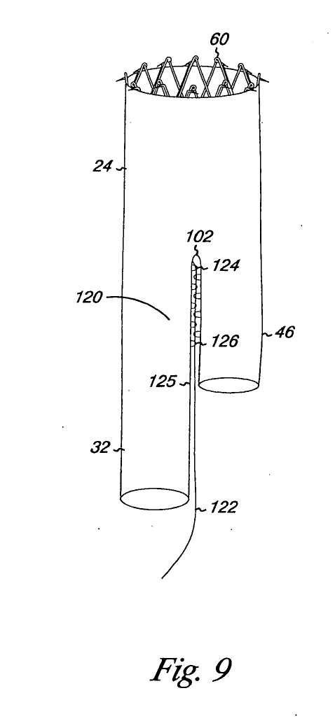

As depicted in FIGS. 9-12, the inferior members or limbs 32, 46 of a modular bifurcated graft may be attached together to improve deployment and post deployment positioning of the endovascular graft within vasculature 202 as well as the in situ assembly of the graft extension 144 to the bifurcated main body 24. If the graft bifurcation 102 is deployed too close to the natural bifurcation of the aneurysm, there is potential that the implant limbs 32, 46 may need to take a sharp bend in order to conform to the aorta anatomy. A sharp bend may kink the limb implant, thereby creating a potential patency issue. Additionally, kinking of the graft 24 may exert stress on the sutures holding graft attachment members together, and may result in suture hole elongation and wear in the graft.

The ipsilateral leg 32 and the contralateral leg stump 46 can be sewn together to improve control, stability, and column stiffness of the graft 24 when accessing the repair site 203. The inferior legs 32, 46 are releasably attached such that the legs are separated after deployment. Sewing the inferior members or limb stumps 32, 46 of the graft together lengthens the effective distance from the top of the aortic graft 24 to the implant bifurcation.

The suture release wire 122 threaded through the suture loops 124 of the bifurcated graft inferior members 32, 46 is withdrawn by pulling an inferior end portion of the suture release wire 122 which can be configured with a pull ring (not shown). Once the suture release wire 122 is removed, the suture attachment mating structure 120 separates the graft limbs 32, 46 allowing the bifurcated graft to conform to anatomy while still providing the necessary control, stability, and column stiffness to the implant during contralateral artery access.

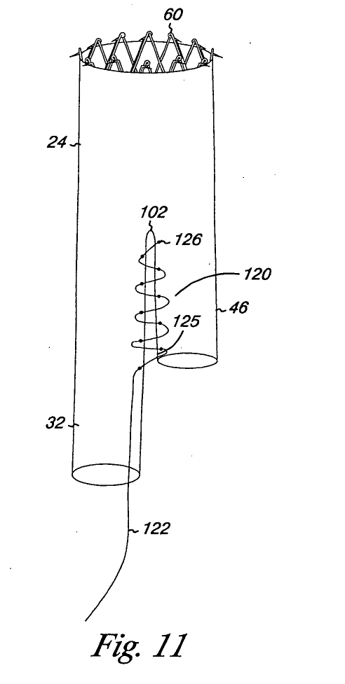

Turning now to FIGS. 9 and 11, there is shown two arrangements for mating or connecting the ipsilateral portion 32 of the graft component 24 to the contralateral graft component 46. With reference to FIG. 9, a first embodiment of mating structure 120 includes a suture 122 that is configured about the inseams of the ipsilateral member 32 and the contralateral member 46 of the graft component 24, such that the members mate or fasten together from the graft bifurcation 102 to an inferior end of the contralateral member 46. The contralateral inferior member 46 can be shorter in length as compared to the ipsilateral member, thereby providing a transplaced effective graft bifurcation 125 while the inferior members 32, 46 are in the mated or connected position. The suture material 122 is configured into a plurality of loops 124 by connecting multiple point locations thereof to the graft component 24 by rings or other suitable means. The mating structure 120 is adapted to define a release interlocking framework securing the ipsilateral and contralateral graft members 32, 46 together.

The suture loop 124 may be made from any flexible substance which is durable and biocompatible. For example, (PET) polyester suture material configured as ties may be suitable for forming the flexible mating members 120.

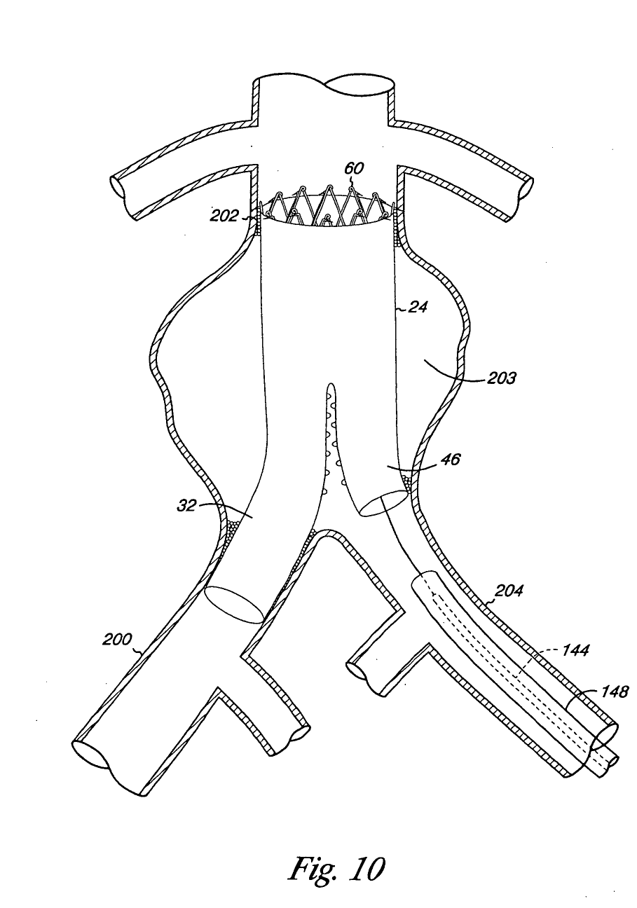

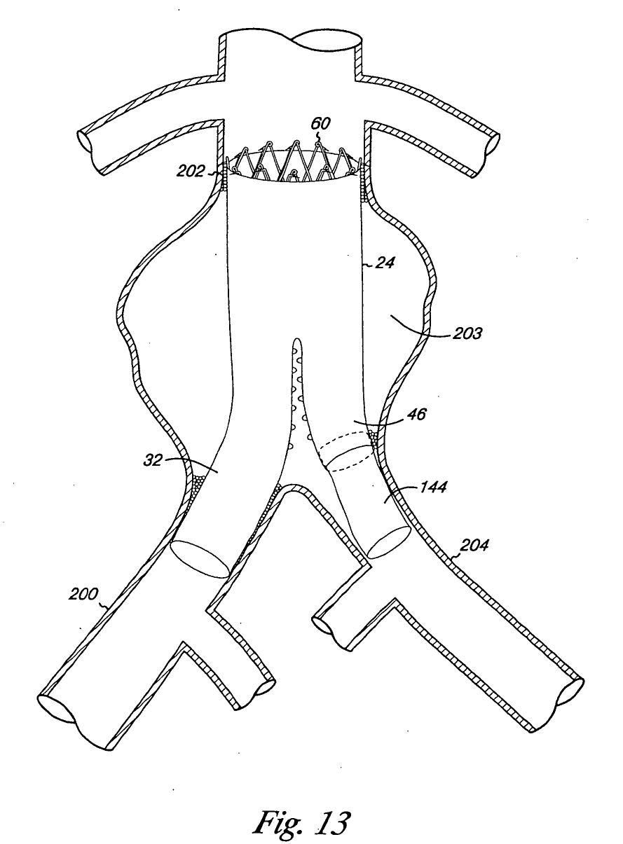

A release wire 122 is threaded through the suture loops 124 affixed to the inseam of the ipsilateral member 32 and the contralateral member 46 to secure the inferior members together (See FIG. 9). The suture release wire 106 also extends proximally throughout the grafting system to an operator or technician. Once the superior attachment mechanism 60 has been securely positioned in an abdominal aorta 203 for example, the remainder of the bifurcated graft 24 may be deployed into the contralateral and ipsilateral branch arteries, as shown in FIG. 10. As depicted in FIG. 10, a contralateral leg extension can be delivered to the graft body and attached to the contralateral leg stump (See FIG. 13).

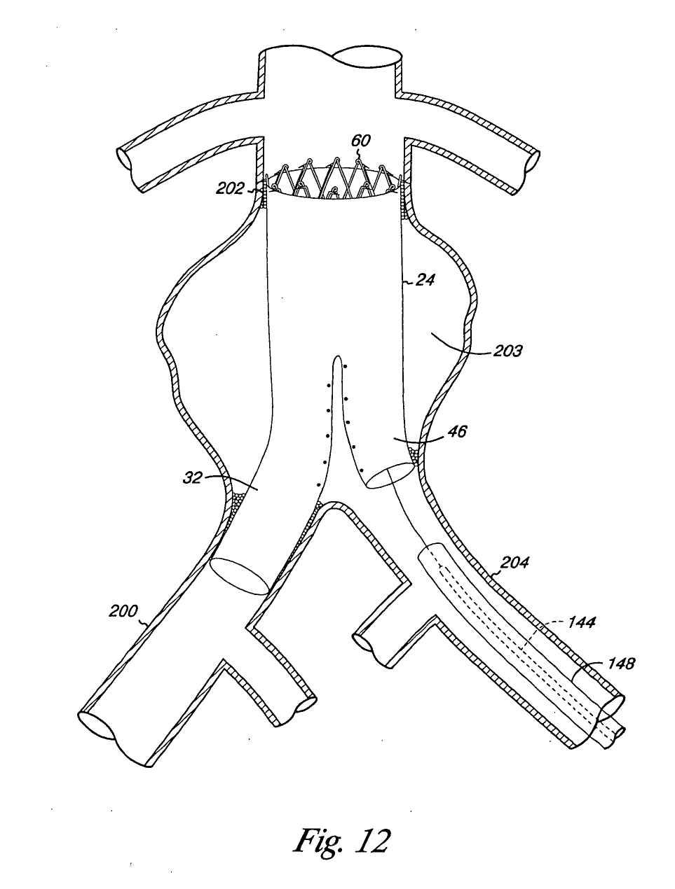

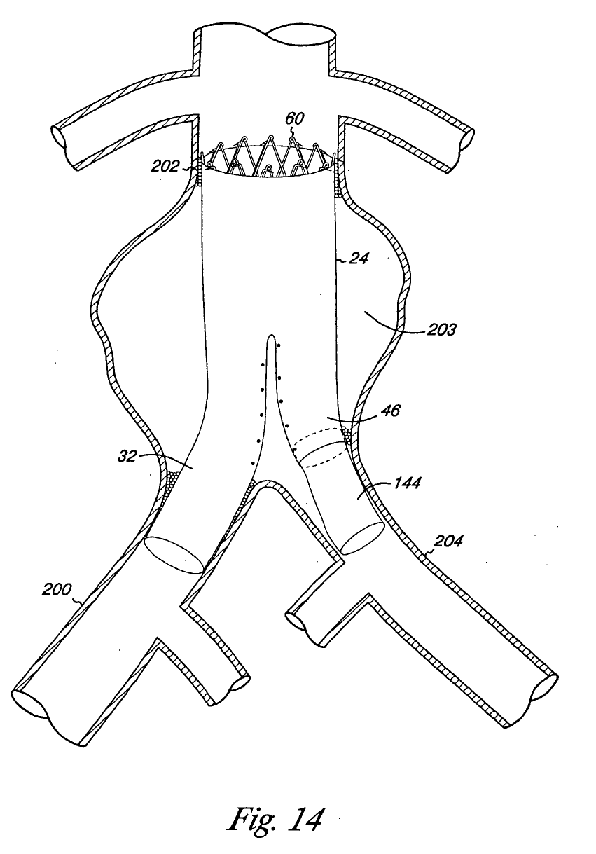

In another preferred embodiment, the mating structure 120 (See FIG. 11) may consist of suture material 126 configured to form a basting or large running stitch pattern which provides temporary attachment that can be easily pulled apart releasing the limb stumps 32, 46. Preferably, the suture material 126 is releasably sewn in a mating pattern from the graft bifurcation or crotch 102, inter-weaving in and out through the ipsilateral member 32 and the contralateral member 46, as shown in FIG. 11. After deployment of the connected graft system, the suture material may be released by pulling and withdrawing the release wire 122. As shown in FIG. 12, after the removal of the suture wire 122, the inferior graft members can be placed within the iliac arteries and the contralateral leg extension may be delivered and installed (See FIG. 14). In this embodiment, the suture material may consist of a biodegradable suture material that would eventually dissolve and release the limb stumps into the anatomy of the aortic aneurysm after deployment.

By way of example, a method for repair of an aortic aneurysm using the present invention for intraluminal placement of a graft in an aorta is described. First, a patient is prepared in a conventional manner by use of a guide wire, a dialator and sheath to access both ipsilateral and contralateral femoral arteries or iliac arteries of the patient. The terminal end of an intraluminal grafting system is then inserted into the sheath, which has previously been placed in the ipsilateral femoral artery. Typically a catheter assembly defines a lumen for receiving the guide wire that is traversed across the aneurysm.

The assemblies may be advanced by the physician as a single unit over a main guide wire. The main guide wire is introduced by the physician into a cutdown in the corporeal lumen and advanced through the ipsilateral iliac artery 200 to the desired location in vasculature 202 and adjacent to the diseased or damaged portion of the vessel 203.

The physician advances the terminal end of the intraluminal grafting system through the ipsilateral femoral artery over the main guide wire. Typically, the desired position for implanting the bifurcated graft 24 will be within the abdominal aorta 203 with the superior extremity of the main tubular member 34 inferior to the renal arteries. Fluoroscopy is used to inspect the position of the main catheter assembly 22 to ensure that the system is not twisted.

Once the superior attachment system 60 has been securely positioned in the abdominal aorta 203, the remainder of the bifurcated graft 24 and delivery system may be exposed. When first exposed, both the contralateral inferior member 46 and the ipsilateral inferior member 32 will be located within the abdominal aneurysm 203.

After being exposed, the contralateral inferior member 46 may be positioned into the contralateral iliac artery 204. A snare loop 104 or similar device is advanced percutaneously or into the cutdown in the contralateral femoral artery. The snare loop is advanced through the contralateral femoral artery and iliac artery. The exposed contralateral guide wire 48 may then be captured (“snared”) by the snare loop, preferably at the hook 146 or bulbous portion formed in the end of the contralateral guide wire 48 which has been placed within the sleeve 100. By withdrawing the snare loop and guide wire 48, the contralateral inferior member 46 can be manipulated via the contralateral guide wire to the desired position of the aorta.

The contralateral inferior member 46 may then be pulled out of the abdominal aorta 203 into the contralateral iliac artery by pulling the contralateral guide wire 48 via the snare loop 104. Should the graft assembly include mating structure 120, the suture release wire 106 can be withdrawn to separate the limbs 32, 46. Once the limbs 32, 46 are positioned as desired, the attachment system 78, 80 may be deployed using conventional apparatus and methods. For example, the attachment systems 78, 80 can be held in a compressed configuration by a release wire or a capsule. Removal of such structure from engagement with the attachment systems 78, 80 allow the same to be implanted within the vasculature. It is to be recognized that while certain figures may depict one of the legs of the bifurcated graft 24 as extending to the iliac arteries, as stated, it is contemplated that graft extensions be employed to bridge the distance from one or both of the bifurcated graft 24 or other tubular graft (FIG. 4) to the iliac arteries. Additionally, the legs 144 can be further extended in the iliac, for example, by additional graft extensions 144. In such an arrangement, terminal ends of the legs of the graft would be configured with structures 78, 80 for mating with other graft components such as graft extensions and to engage the vessel wall 202.

Once the graft 24 is implanted at the repair site, the various components used to deploy the system are removed. For example, by pulling the snare loop and guide wire proximally, the physician removes these components through the contralateral iliac and femoral arteries.

It is to be noted that either before or after the positioning and securing of the contralateral inferior member 46, the ipsilateral inferior member 32 may be positioned and secured. Once the ipsilateral inferior member is in place, the ipsilateral attachment system 78 may be deployed. Additionally, the contralateral member 32 can be mated with other graft components delivered through the contralateral catheter 148.

The entire procedure described herein can be observed under fluoroscopy. The relative positioning of the bifurcated graft 24 can be readily ascertained by the radiopaque markers 116 provided on the graft, and the radiopaque markers 116 on the sleeve 100 or the radiopaque inferior attachment systems themselves. If any twisting of the graft has occurred between placement of the superior attachment system and the inferior attachment systems then the twisting can be readily ascertained by observing markers. Adjustments to eliminate any twisting which may have occurred can be made before exposing the attachment systems. Any excessive graft compression may also be ascertained by observing the radiopaque markers under fluoroscopy.

Post implant fluoroscopy procedures may be utilized to confirm the proper implantation of the device by the use of a conventional pigtail catheter or by injecting dye into the guide wire lumen of the balloon catheter shaft. Thereafter the sheath can be removed from the femoral artery and the femoral artery closed with conventional suturing techniques. As described above, a blood tight seal at the three attachment sites establish a complete repair of the vessel. Thereafter, tissue may begin to grow into or over the graft within two to four weeks with tissue covering the interior side of the graft within six months. Moreover, blood-tight seals are provided at the three attachment sites by the cooperation of the attachment systems and the graft to thereby accomplish a complete repair.

While several particular forms of the invention have been illustrated and described, it will be apparent that various modifications can be made without departing from the spirit and scope of the invention. For example, references to materials of construction and certain dimensions are also not intended to be limiting in any manner and other materials and dimensions could be substituted and remain within the spirit and scope of the invention. Accordingly, it is not intended that the invention be limited, except as by the appended claims.

What is claimed:

1. An endovascular graft system, comprising:
a graft body;
an elongate positioning mechanism, the positioning mechanism facilitating intraluminally positioning the graft into a corporeal lumen; and
a sleeve attached to the graft body, the sleeve configured to route the elongate positioning mechanism through the graft.

2. The system of claim 1, the positioning mechanism includes a guide wire.

3. The system of claim 1, wherein the graft is bifurcated defining a superior member, an ipsilateral member and a contralateral member, and wherein the sleeve extends from the ipsilateral member of the bifurcated graft to the contralateral member.

4. The system of claim 2, wherein the sleeve is disposed about the guide wire prior to deployment into the corporeal lumen.

5. The system of claim 1, the elongate positioning mechanism further including a snaring means, wherein the snaring means is configured to engage a snare device.

6. The system of claim 1, wherein the sleeve is formed of a flexible surgical implantable material and affixed to the graft body with at least one suture or is woven into the graft body or thermally bonded thereto.

7. The system of claim 1, wherein the sleeve includes at least one radiopaque marker for positioning the graft body within vasculature.

8. The system of claim 1, the sleeve further including a pressure sensing means.

9. A system for treating a body lumen, comprising:
a graft body;
an attachment system capable of intraluminally attaching a graft to a vessel wall, the attachment system including a frame configured to be affixed to the graft body and to assert an outwardly directed bias, the frame having a plurality of wall engaging members when the attachment system is expanded; and
a graft pocket formed in the graft body, the graft pocket being responsive to pressure provided by blood flow, the graft pocket acting as a sealing member.

10. The system of claim 9, the graft pocket forming a circumferential seal against the vessel wall.

11. The system of claim 7, the graft pocket having a diameter in the range of 1 mm to 6 mm larger than a diameter of the graft body.

12. The system of claim 9, the attachment system wire frame is not attached to the graft pocket thereby allowing the graft pocket to expand independent of the attachment system.

13. The system of claim 9, the system including a plurality of graft pockets configured along the graft body.

14. The system of claim 12, wherein the attachment system is affixed to the graft body via a stitching pattern having at least two double loop knots and at least two suture loops around the attachment system, and a running stitch having threaded loops and double loop knots.

15. An endovascular graft, comprising:
a bifurcated graft formed of a superior member having a graft bifurcation and extending into an ipsilateral member and a contralateral member; and
a mating structure that releasably attaches the ipsilateral member and the contralateral members of the bifurcated graft, wherein the members are in the attached position during deployment and separated after post deployment positioning of the graft into the corporeal lumen.

16. The graft of claim 15, wherein the mating structure includes a plurality of suture loops affixed about an exterior inseam of the ipsilateral member and the contralateral member.

17. The graft of claim 16, wherein the mating structure further includes a release wire that is releasably threaded through the suture loops to secure the ipsilateral and contralateral members together, wherein the removal of the release wire separates the graft members allowing the bifurcated graft to conform to a bifurcation.

18. The graft of claim 15, the mating structure having a suture material releasably configured to form a running stitch pattern that is sewn to attach the ipsilateral member and contralateral member.

19. The mating structure of claim 18, wherein the suture running stitch begins at the graft bifurcation and is stitched in-and-out through the ipsilateral member and contralateral member.

20. An endovascular graft system, comprising:
a graft body;
a sleeve attached to the graft body;
an expandable attachment system capable of intraluminally attaching the graft body to a vessel; and
a sealing member configured to radially surround the graft body.

21. The system of claim 20, further comprising a guide wire configured to be received by the sleeve.

22. The system of claim 20, wherein the graft body is bifurcated and defines an ipsilateral member and a contralateral member, and wherein the sleeve starts on the ipsilateral member of the bifurcated graft and ends on the contralateral member.

23. The system of claim 20, wherein the sleeve is disposed about the guide wire prior to deployment into vasculature.

24. The system of claim 21, the guide wire having a terminal end including a hook.

25. The system of claim 24, further comprising a snare device configured to engage the hook.

26. The graft of claim 22, wherein the sleeve is configured of a flexible surgical implantable material, and affixed to the graft body with at least one suture or is woven into the graft body or thermally bonded thereto.

27. The graft of claim 22, wherein the sleeve is affixed to the to the graft body with a plurality of sutures.

28. The graft of claim 20, the sealing member includes a graft pocket configured to form a leak tight seal, wherein the graft pocket expands radially with induced fluid pressure forming a circumferential seal and redirects fluid flow into the graft.

29. The system of claim 20, wherein the attachment system further includes a plurality of wall-engaging members.

30. The system of claim 20, wherein the attachment system is attached to the superior end of the graft body.

31. An endovascular graft for repairing a blood vessel, comprising:
a graft body having a plurality of openings;
an expandable attachment system capable of intraluminally attaching a superior opening to a vessel; and
a sealing member radially affixed to the graft member attachment site to prevent blood leakage at the attachment site, the sealing member defined by tufted material formed of a suture that is stitched circumferentially in an in-and-out pattern forming suture loops around a circumference of the graft body.

32. The sealing member of claim 31, the suture material is polyethyleneterephthalate.

33. An endovascular graft for repairing a blood vessel, comprising:
a graft body having a plurality of openings;
an expandable attachment system capable of intraluminally attaching a superior opening to a vessel; and
a sealing member radially affixed to the graft member attachment site to prevent blood leakage at the attachment site, the sealing member defined by tufted material is fabric formed from a non-woven web of loose fibers attached to the graft member walls by a suture thread, wherein the non-woven web has an in-air thickness of approximately 0.01 in. and a compressed thickness in the range of 0.007 in. to 0.008 in., and a width of approximately 5 cm.

34. The sealing member of claim 33, the suture material is polyethyleneterephthalate.

35. An endovascular graft, comprising:
a bifurcated graft formed of a superior member having a graft bifurcation and extending into an ipsilateral member and a contralateral member; and
a mating structure that releasably attaches the ipsilateral member and the contralateral members of the bifurcated graft, wherein the members are in the attached position during deployment and separated after post deployment positioning of the graft into the corporeal lumen.

36. The graft of claim 35, wherein the mating structure includes a plurality of suture loops affixed about an exterior inseam of the ipsilateral member and the contralateral member, wherein the suture material is biocompatible and flexible.

37. The graft of claim 36, the mating structure further includes a release wire that is releasably threaded through the suture loops to secure the ipsilateral and contralateral members together, wherein the removal of the release wire separates the graft members allowing the bifurcated graft to conform to an aortic bifurcation.

38. The graft of claim 35, wherein the mating structure includes a suture material releasably configured to form a running stitch pattern that is sewn to attach the ipsilateral member and contralateral member.

39. The mating structure of claim 38, wherein the suture running stitch begins at the graft bifurcation and is stitched in-and-out through the ipsilateral member and contralateral member.

40. The graft of claim 38, further comprising a graft pocket.
